(19) United States
(12) Patent Application Publication
(10) Pub. No.: US 2011/0118353 A1
(43) Pub. Date: May 19, 2011

(54) METHOD OF HORMONE SUPPRESSION IN HUMANS

(75) Inventors: Robert Gerard Jules Marie Hanssen, Oss (NL); Jacques Schipper, Oss (NL); Josephus Hubertus Schoemaker, Oss (NL)

(73) Assignee: N. V. Organon

(21) Appl. No.: 12/740,716

(22) PCT Filed: Nov. 4, 2008
(86) PCT No.: PCT/EP2008/064914

§ 371 Date: Dec. 2, 2010

(30) Foreign Application Priority Data
Nov. 6, 2007 (EP) 07120100.8

(51) Int. Cl. A61K 31/198; C07C 229/36; A61P 5/26; A61P 5/30; A61P 5/24; A61P 15/00

(52) U.S. Cl. 514/567; 562/443; 562/444; 562/449

(57) ABSTRACT

The present invention relates to a glycine transporter-1 inhibitor having the formula (I) wherein X is 1-3 substituents selected from H, halogen, methyl, methoxy, trifluoromethyl and trifluoromethoxy and Y is 1-3 substituents selected from H, methyl and halogen or a pharmaceutically acceptable salt thereof for use in a treatment in humans to suppress the level of one or more hormone selected from luteinizing hormone, follicle-stimulating hormone, estradiol and testosterone. The present invention further relates to such a glycine transporter-1 inhibitor as part of a contraceptive regimen or as a treatment for hypersexuality.

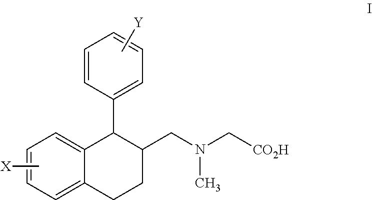

The present invention relates to a a glycine transporter-1 inhibitor for use in hormone suppression in humans. More specifically, the present invention relates to a glycine transporter-1 inhibitor for use in a treatment in humans to suppress the level of one or more hormone selected from luteinizing hormone, follicle-stimulating hormone, estradiol and testosterone. The present invention further relates to a glycine transporter-1 inhibitor for use in the treatment or prevention in humans of a disease or disorder associated with an adverse level of one or more hormone selected from luteinizing hormone, follicle-stimulating hormone, estradiol and testosterone.

Luteinizing hormone (LH) is a small glycoprotein hormone secreted by the anterior pituitary gland. LH plays an important role in controlling ovulation and in controlling synthesis and secretion of hormones by the ovaries and testes. Follicle-stimulating hormone (FSH) is a gonadotrophic glycoprotein hormone also found in the anterior pituitary gland of mammals. It stimulates ovarian granulosa cells and testicular sertoli cells, induces maturation of Graafian follicles in the ovary and promotes the development of the germinal cells in the testes. In response to stimulation by LH in the anterior pituitary, testosterone is produced by the interstitial (Leydig) cells of the testes.LH and FSH stimulate, in concert, estradiol production in iovarian granulosa cells. LH, FSH, estradiol and testosterone therefore play an important part in human sexual function.

Since their introduction over forty years ago, oral contraceptives have found widespread application in the regulation of female fertility. Available therapies have changed over time with the discovery of new estrogens and progestagens, the development of progestagen only regimes and the introduction of combined progestagen/estrogen regimes with reduced estrogen content. In spite of these advances providing oral contraceptives with an improved safety profile relative to earlier therapies, unwanted side-effects still persist. In particular, adverse metabolic effects caused by the estrogen component and possible neoplastic effects can result. Accordingly, there remains a need for new oral contraceptive therapies, in particular, non-steroidal and non-hormonal contraceptives which are safe as well as effective. For a recent review see Current. Pharm Design, 2006, 12(30), 3915-28.

Hypersexuality or compulsive sexual behaviour remains a disorder for which there is also a need for further treatment regimes. Antidepressants or naltrexone have been used to reduce anxiety or depression often associated with sexual obsession. There exists therefore a need for further therapies for hypersexuality which are both safe and effective.

In a first aspect, the present invention provides a glycine transporter-1 (GIyT1) inhibitor having the formula I

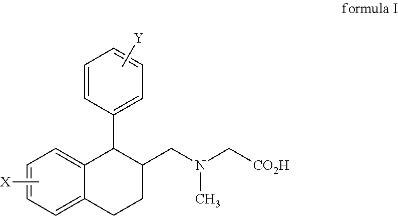

wherein
X is 1-3 substituents selected from H, halogen, methyl, methoxy, trifluoromethyl and trifluoromethoxy and
Y is 1-3 substituents selected from H, methyl and halogen
   or a pharmaceutically acceptable salt thereof
   for use in a treatment in humans to suppress the level of one or more hormone selected from LH, FSH, estradiol and testosterone. The present invention therefore provides a method of suppression of one or more of LH, FSH, estradiol and testosterone in humans comprising administering an effective amount of a GlyT1 inhibitor having the formula I, wherein X and Y have the previously defined meanings, to a subject in need thereof.

The term halogen, as used herein, represents a fluorine, chlorine, bromine or iodine.

In one embodiment of the present invention is a GlyT1 inhibitor having the formula I, wherein X and Y have the previously defined meanings, for use in a treatment in humans to lower the level of LH.

In a further embodiment of the present invention is a GlyT1 inhibitor having the formula I, wherein X and Y have the previously defined meanings, for use in a treatment in humans to lower the level of FSH.

In a further embodiment of the present invention is a GlyT1 inhibitor having the formula I, wherein X and Y have the previously defined meanings, for use in a treatment in humans to lower the level of estradiol.

In a further embodiment of the present invention is a GlyT1 inhibitor having the formula I, wherein X and Y have the previously defined meanings, for use in a treatment in humans to lower the level of testosterone.

In a further aspect of the present invention, a GlyT1 inhibitor having the formula I, wherein X and Y have the previously defined meanings, for use in a treatment in humans to suppress the level of one or more hormone selected from LH, FSH, estradiol and testosterone forms part of a contraceptive regimen. In a further embodiment of the present invention, the contraceptive regimen is for male contraception. In a further embodiment of the present invention, the contraceptive regimen is for female contraception.

In a further aspect of the present invention, a GlyT1 inhibitor having the formula I, wherein X and Y have the previously defined meanings, is useful in the treatment of hypersexuality in humans, wherein said treatment involves suppression of the level of one or more hormone selected from LH, FSH, estradiol and testosterone.

In a further aspect of the present invention, a GlyT1 inhibitor having the formula I, wherein X and Y have the previously defined meanings, is useful in the treatment of aggression in humans, wherein said treatment involves suppression of the level of one or more hormone selected from LH, FSH, estradiol and testosterone.

In a further aspect of the present invention, a GlyT1 inhibitor having the formula I, wherein X and Y have the previously defined meanings, is useful in the treatment in humans of a disease or disorder selected from hirsutism, excess sebum production, breast cancer, benign breast disease, benign ovarian disease, polycystic ovarian disease, endogeneous LH surges in controlled ovarian stimulation in fertility treatment, miscarriage associated with excess androgen, benign prostatic hyperplasia, prostate cancer, endometriosis or uterine fibroids, uterus leiomyoma, uterus leiomysarcoma, hyperandrogenism, oligomenorrhoea and hair loss, wherein said treatment involves suppression of the level of one or more hormone selected from LH, FSH, estradiol and testosterone.

Reuptake of glycine via glycine transporter (GlyT) proteins into presynaptic nerve terminals or neighbouring glial cells constitutes an effective mechanism by which the postsynaptic actions of glycine can be terminated and extracellular glycine levels returned to basal values. There are today two known types of glycine transporter proteins, the glial transporter (type 1), GlyT1, and the glycine neural transporter (type 2), GlyT2. The GlyT1 catalyses the removal of glycine from the synaptic cleft and the GlyT2 is required for the reuptake and reloading of glycine into the synaptic vesicle (Gomeza et al., 2003; Curr Opin Drug Discov Devel 6(5): 675-82).

Many different structural classes of compounds which act as inhibitors of glycine transporter proteins are known in the art. Compounds which are selective inhibitors of GlyT1 or GlyT2 are also known. See L. G. Harsing Jr. et al., Current Med. Chem., 2006, 13, 1017-44 and S. M. Lechner, Current Opinion in Pharmacology, 2006, 6(1), 75-78 for recent reviews showing examples of compounds which act as selective GlyT1 inhibitors. Previously, such GlyT1 inhibitors have been suggested to find an application in the treatment of disorders such as schizophrenia, depression, dementia and other forms of impaired cognition, neurodegenerative diseases or muscle hyperactivity associated with spasticity, myoclonus and epilepsy.

Methods for the preparation of GlyT1 inhibitors having the formula I

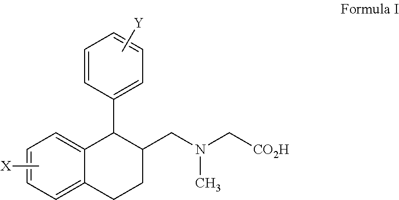

wherein X and Y have the previously defined meanings are described in WO 00/07978.

The present invention also includes within its scope use of all stereoisomeric forms of the GlyT1 inhibitors of formula I, wherein X and Y have the previously defined meanings resulting, for example, because of configurational or geometrical isomerism. Such stereoisomeric forms are enantiomers, diastereoisomers, cis and trans isomers etc. In the case of the individual enantiomers of compounds of formula I or salts thereof, the present invention includes use of the aforementioned stereoisomers substantially free, i.e., associated with less than 5%, preferably less than 2% and in particular less than 1% of the other enantiomer. Use of mixtures of stereoisomers in any proportion, for example a racemic mixture comprising substantially equal amounts of two enantiomers are also included within the scope of the present invention.

In a further embodiment of the present invention is a GlyT1 inhibitor is selected from:
N-methyl-N-[(1R,2S)-1,2,3,4-tetrahydro-6-methyl-1-phenyl-2-naphthalenyl]methyl glycine;
N-methyl-N-[(1R,2S)-1,2,3,4-tetrahydro-6-methoxy-1-phenyl-2-naphthalenyl]methyl glycine;
N-methyl-N-[(1R,2S)-1,2,3,4-tetrahydro-6-ethyl-1-phenyl-2-naphthalenyl]methyl glycine;
N-methyl-N-[(1R,2S)-1,2,3,4-tetrahydro-6-ethoxy-1-phenyl-2-naphthalenyl]methyl glycine;
N-methyl-N-[(1R,2S)-1,2,3,4-tetrahydro-6-trifluoromethyl-1-phenyl-2-naphthalenyl]methyl glycine and
N-methyl-N-[(1R,2S)-1,2,3,4-tetrahydro-6-trifluoromethoxy-1-phenyl-2-naphthalenyl]methyl glycine
   or a pharmaceutically acceptable salt thereof
   for use in a treatment in humans to suppress the level of one or more hormone selected from LH, FSH, estradiol and testosterone.

In a further embodiment of the present invention, the GlyT1 inhibitor can be combined with a known contraceptive agent. This has the advantage of providing a means of contaception with a lower burden of estrogenic or progestagenic or androgenic side-effects. Hence in a further embodiment of the present invention is a GlyT1 inhibitor having the formula I, wherein X and Y have the previously defined meanings, for use in a treatment in humans to suppress the level of one or more hormone selected from LH, FSH, estradiol and testosterone, wherein said GlyT1 inhibitor forms part of a contraceptive regimen which comprises an estrogen as a further active component.

In a still further embodiment of the present invention is a GlyT1 inhibitor having the formula I, wherein X and Y have the previously defined meanings, for use in a treatment in humans to suppress the level of one or more hormone selected from LH, FSH, estradiol and testosterone, wherein said GlyT1 inhibitor forms part of a contraceptive regimen which comprises a progestagen as a further active component.

In a still further embodiment of the present invention is a GlyT1 inhibitor having the formula I, wherein X and Y have the previously defined meanings, for use in a treatment in humans to suppress the level of one or more hormone selected from LH, FSH, estradiol and testosterone, wherein said GlyT1 inhibitor forms part of a contraceptive regimen which comprises an androgen as a further active component.

Pharmaceutical compositions for the use as claimed and described herein can be prepared in accordance with standard techniques in the art of pharmaceutical sciences. The compounds can be used for humans in a dosage of 0.001-50 mg per kg body weight, preferably in a dosage of 0.01-20 mg per kg body weight, whereby the optimum dosage can be determined according to factors such as route of administration, desired duration of action, type of formulation (extended release versus immediate release) type of patient, type of compound required, efficacy of the compound and other physical characteristics of the recipient of the treatment, such co-morbidity of other diseases, liver metabolism capacity, etc.

Selective transport inhibition and methods how to determine such a biological effect can be determined according to known techniques in the biochemistry of glycine. A specific method is described in the example below, on which basis a criterion pIC₅₀ value of at least 6.0, or preferably 6.5, or even better 7.0 can be derived for clarity of the meaning of the term glycine transport type 1 inhibitor.

The present invention is illustrated by the following examples which are in not intended to limit the scope thereof:

### EXAMPLE 1

### Method for Determination of Glycine Uptake in CHO Cells Heterologously Expressing the Human GlyT-1b Transporter

A: Cloning: cDNA was generated by PCR according to the method described by Kim, K.-M. et al. Mol. Pharmacol. 1994, 45, 608-617. Sequence was verified by dideoxy sequencing using the ALF DNA sequencer™ (Pharmacia) and cloned into the expression construct pcDNA3 (Invitrogen).

B: Transfection: Transfection of hGlyT-1b into CHO cells was performed using a standard calcium phosphate technique as described by Sambrook, J. et al. (1989) in Molecular Cloning: A Laboratory Manual, Cold Spring Harbor Laboratory, Cold Spring Harbor, N.Y.

C: Selection: Stably transfected cells were selected for 1 week in growth medium containing 1 mg.cm⁻³ Geneticin. Individual clones were picked for further analysis and positives passaged routinely as described below.

D: Culture conditions: Cells stably expressing the hGlyT-1b gene were cultured at 37° C. in a 5% CO₂ atmosphere in DMEM—NUT.MIX. F12 with Glutamax-1 (Gibco) containing Geneticin (0.5mg.cm⁻³, Gibco) and supplemented with 10% Fetalclone II (Hyclone). Maintenance culture was carried out in standard 80 cm² ventilated flasks (2×10⁻⁶ m filter, Nunc) and cells were subcultured by trypsinisation (Sigma) when confluent.

E: Assay Procedure: Cells for uptake studies were plated in 96 well plates (17,000 cells per well) in the absence of Geneticin and cultured for 48 h before use. To measure glycine transport, cells were washed twice with Hanks' balanced salt solution (HBSS) pre-warmed to 37° C. and excess fluid removed before addition of test compounds dissolved in 0.200 cm³ HBSS. Plates were incubated at 37° C. for 5 minutes before addition of [³H]glycine (0.050 cm³, 150×10⁻⁶ M, 248 Bq.nmol⁻¹, NEN) and incubation continued for a further 10 minutes. Uptake was terminated by washing cells with ice-cold HBSS before removal of excess fluid and addition of 0.200 cm³ scintillation cocktail to each well. Plates were sealed with adhesive film, shaken to ensure samples were homogenous before scintillation counting in a plate counter.

F: Data Analysis: Data were analysed using standard curve fitting procedures to produce a pIC₅₀ value for active compounds (where pIC₅₀ is the negative logarithm of the concentration of test compound causing 50% inhibition of uptake).

G: Result:

The pIC₅₀ values of compounds meant to be glycine transport type 1 inhibitors in this description are those having a pIC₅₀ value of at least 6.0.

### EXAMPLE 2

### Method of Suppression of LH, FSH and Testosterone Upon Administration of N-methyl-N-[[(1R,25)-1,2,3,4-tetrahydro-6-methoxy-1-phenyl-2-naphthalenyl]methyl Glycine Hydrochloride (Compound 1) to Male Subjects

### Experiment 1

A double-blind, cross-over, placebo controlled, single rising oral dose trial with compound 1 was carried out in healthy male volunteers to assess its tolerability, safety, pharmacokinetic and pharmacodynamic profile.

### Trial Design

This was a double-blind, placebo-controlled, single rising oral dose study in 16 subjects. Each subject was assigned to one of 4 dosing groups and received 3 successive single oral doses of compound 1 and placebo in a four-way cross-over design. Subjects were male volunteers with a good physical and mental health, aged 18-45 years, body mass index 18-28 kg/mg².

### Treatments

For this trial a freeze dried cake of compound 1 (batch No. CV 195 and PW 037) was reconstituted with water for injection and diluted with gelatin/mannitol to 50 mL solution for oral administration. Dose levels of compound 1 used in this trial were:

Group 1: 0.5, 1 and 2 mg compound 1

### Group 2: 2, 3 and 5 mg

### Group 3: 5, 8 and 12 mg

### Group 4: 12, 20 and 30 mg

### Blood Sampling

Serum samples for LH, FSH and testosterone analysis were taken at time points: pre-dose and 1, 2, 3, 4, 6, 8 and 12 hours post dose. Serum blood was stored at −40° C.

### Bio-Analytical Methods

LH and FSH were measured using a DELFIA assay. The DELFIA LH and DELFIA FSH assays are solid phase, two-site fluoroimmunometric assays based on the direct sandwich technique. Testosterone was measured using a DELFIA testosterone assay; a solid phase fluoro-immuno-assay based on competition between Europium-labeled Testosterone and sample Testosterone. The specificity of the assays against compound 1 was tested on a concentration level of 50 ng compound 1 per mL serum, to prove that there is no influence of compound 1 on the immuno response of the testkit.

### Statistical Methods

Effect of treatment was characterised by calculating areas under the effect curve (AUE) over 0-4 h and 0-final assessment time periods. AUEs were calculated with the linear trapezoidal rule using protocol times. The pre-value was set at t=0 h. AUEs were divided by the corresponding time span, resulting in a weighted average response. All measures were analysed using analysis of variance with factors subject and treatment and with the prevalue as covariate. A linear contrast on dose group was calculated with dose groups assigned in ascending order.

### Results:

A statistically significant treatment effect was found for LH levels from 0 to 12 hours post-dose (i.e., a decresae in LH levels). Both FSH and testosterone responded slower than LH-concentrations displaying significant linear decreases with rising doses, for the 0-12 h period.

### Experiment 2

A single-dose, double-blind, placebo-controlled, randomized, crossover trial to evaluate visual effects of compound 1 in healthy male volunteers

### Trial Design:

This was a double-blind, placebo-controlled, four-period crossover study involving the administration of three single oral doses of compound 1 and placebo in 24 healthy male subjects. For each subject the compound 1 treatments were randomized in ascending order, each treatment period was separated by at least 3 days wash-out. Subjects were male volunteers with a good physical and mental health, aged 18-45 years, body mass index 18-28 kg/m².

### Treatments:

A freeze dried cake of compound 1 (Batch No CW122) was reconstituted with de-ionized water BP and subsequently diluted with gelatin/mannitol to 50 mL solution for oral administration. Dose levels of compound 1 used in this trial were: 5 mg, 13 mg and 20 mg

### Blood Sampling:

Serum samples for LH, FSH and testosterone analysis were taken at time points: pre-dose and 20′, 45′, 1h10′, 1h35′, 2h, 2h25′, 2h50′, 3h15′, 4h, 6h, 8h, 12h, 16h and 24h post-dose. Serum was stored at −40° C.

### Bio-Analytical Methods:

LH and FSH were measured using a DELFIA assay. The DELFIA LH and DELFIA FSH assays are solid phase, two-site fluoroimmunometric assays based on the direct sandwich technique. Testosterone was measured using a DELFIA testosterone assay; a solid phase fluoro-immuno-assay based on competition between Europium-labeled Testosterone and sample Testosterone. The specificity of the assays against compound 1 was tested on a concentration level of 50 ng compound1 per mL serum, to prove that there is no influence of compound 1 on the immuno response of the testkit.

### Statistical Methods

Descriptive statistics for serum FSH, LH and testosterone concentrations were calculated, both as absolute values and as percentage of baseline. In addition the following parameters were calculated: Cₘᵢₙ, tₘᵢₙ, C_{mean,0-24}, both Cₘᵢₙ and C_{mean,0-24} were expressed in absolute values and as percentage of baseline. No formal statistical analysis was done on these PD parameters. Only descriptive statistics were calculated by dose. Furthermore, summary plots of the PD parameters versus treatment were made

### Results:

LH and testosterone concentrations decrease after compound 1 treatment, where decreases become larger at increasing doses of compound 1. The largest LH decrease is reached approximately 4 h post dose, the effect on testosterone seems somewhat slower. An effect of compound 1 on FSH concentrations is not clearly visible. The adjusted Cₘᵢₙ values for FSH, LH and Testosterone show a decrease with increasing active dose. The adjusted Cₘₑₐₙ is less sensitive to detect effects of compound 1 treatment, decreases are smaller and less consistent.

### Experiment 3

A phase I, double-blind, placebo-controlled, parallel group, multiple oral dose and multiple ascending dose study with compound 1 in healthy male volunteers to assess the safety, tolerability, pharmacokinetics and pharmacodynamics of compound 1 after single, multiple and multiple ascending doses.

### Trial Design:

This was a single center, double-blind, placebo-controlled, parallel group design. A total of 40 subjects were randomized over 5 groups of 8 subjects; within each group 6 subjects received multiple oral doses of compound 1 and 2 subjects received placebo. Subjects were male volunteers with a good physical and mental health, aged 18-45 years, body mass index 18-28 kg/m².

### Treatments:

Freeze dried cake of compound 1 (Batch No. CW 186) was supplied in 10 mL vials (50 mg active entity in 10 mL vials). The freeze dried cake was reconstituted with sterile de-ionized water B.P and subsequently diluted with sterile de-ionized water to a dose volume of 50 mL. Compound 1 was administered as an oral solution (50 mL) according to the following schedule:
Group 1: a single dose of 4 mg compound 1 on day 1 followed by once daily dosing of 4 mg compound 1 on days 4 to 13
Group 2: a single dose of 8 mg compound 1 on day 1 followed by once daily dosing of 8 mg compound 1 on days 4 to 13
Group 3: a single dose of 16 mg compound 1 on day 1 followed by once daily dosing of 16 mg compound 1 on days 4 to 13
Group 4: a single dose of 12 mg compound 1 on day 1, twice daily dosing of 12 mg compound 1 on days 4 to 12 followed by a single dose of 16 mg compound 1 on day 13

For group 5 a dose titration was used. In this group no samples for LH, FSH and testosterone were taken.

### Blood Sampling:

Serum samples for LH, FSH and testosterone analysis were taken at time points: on days 1, 3, 6, 8, 10 and 13: pre-dose, 2, 6 and 12 h post-dose; on day 15: 48 h post-dose (samples taken on day 15 but time points relative to dosing on day 13). Serum was stored at −40° C. No blood samples were taken for group 5.

### Bio-Analytical Methods:

LH and FSH were measured using a DELFIA assay. The DELFIA LH and DELFIA FSH assays are solid phase, two-site fluoroimmunometric assays based on the direct sandwich technique. Testosterone was measured using a DELFIA testosterone assay; a solid phase fluoro-immuno-assay based on competition between Europium-labeled Testosterone and sample Testosterone. The specificity of the assays for compound 1 was tested on a concentration level of 50 ng compound 1 per mL serum, to prove that there is no influence of compound 1 on the immuno response of the testkit.

### Statistical Methods:

No formal statistical analysis was. Serum LH, FSH and testosterone concentrations were listed and summarized for each dose group and time point. In addition, changes from baseline (Day 1 predose) were listed and summarized. Mean plots of changes from baseline against time were produced by dose group

### Results:

Mean FSH and LH values decreased following dosing and the magnitude of decrease was generally largest following administration of 16 mg compound 1. The maximum decrease in FSH values was seen at approximately 6 to 12 h post-dose, the maximum decreases in LH occurred at 2 hours after dosing. Testosterone levels dropped sharply after dosing until 6-12 h post-dose after which levels rose again to approximately baseline levels following all treatments. Whilst this was at least in part due to the circadian rhythm of testosterone, the magnitude of the decrease was generally larger following administration of compound 1 with the largest decreases in the 16 mg q.d. and 12 mg b.i.d groups. For FSH, LH, and testosterone decreases from baseline were similar on day 13 and on day 1 for most treatment groups.

### Experiment 4

A phase I, double-blind, parallel groups, placebo-controlled, cross-over, pharmacodynamic study with compound 1 in healthy male volunteers to assess glycinergic responses in cerebro-spinal fluid and plasma after single oral doses

### Trial Design:

This was a double-blind, placebo-controlled, parallel group trial in 15 subjects, randomized to 3 parallel groups of 5 subjects each. The subjects received two single doses in a randomized cross-over design. There was an interval of at least 3 days between the drug administrations. Subjects were male volunteers with a good physical and mental health, aged 18-45 years, body mass index 18-28 kg/m².

### Medication:

A freeze dried cake of compound 1 (Batch No CW122) was reconstituted with sterile water and subsequently further diluted with orange juice to a total volume of 200 mL for oral administration. Dose levels used were:
Group 1: 8 mg compound 1 and glycine
Group 2: 16 mg compound 1 and placebo
Group 3: 8 mg compound 1 and 4 mg compound 1

### Blood Sampling:

Serum samples for LH, FSH and testosterone analysis were taken at time points: pre-dose and 1, 2, 3, 4, 6 and 12 hours post dose. Serum was stored at −40° C.

### Bio-Analytical Methods:

LH and FSH were measured using a DELFIA assay. The DELFIA LH and DELFIA FSH assays are solid phase, two-site fluoroimmunometric assays based on the direct sandwich technique. Testosterone was measured using a DELFIA testosterone assay; a solid phase fluoro-immuno-assay based on competition between Europium-labeled Testosterone and sample Testosterone. The specificity of the assays against compound 1 was tested on a concentration level of 50 ng compound 1 per mL serum, to prove that there is no influence of compound 1 on the immuno response of the testkit.

### Results:

LH and testosterone were suppressed to some extent after compound 1 treatment. No effect on FSH was present.

1-11. (canceled)

12. A glycine transporter-1 inhibitor having the formula I 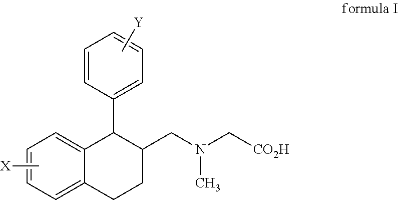
wherein
X is 1-3 substituents selected from H, halogen, methyl, methoxy, trifluoromethyl and trifluoromethoxy and
Y is 1-3 substituents selected from H, methyl and halogen
or a pharmaceutically acceptable salt thereof.

13. The glycine transporter-1 inhibitor according to claim 1 which is N-methyl-N-[[(1R,2S)-1,2,3,4-tetrahydro-6-methoxy-1-phenyl-2-naphthalenyl]methyl glycine or a pharmaceutically acceptable salt thereof.

14. A pharmaceutical composition comprising the compound according to claim 12 or a pharmaceutically acceptable salt thereof and a pharmaceutically acceptable carrier.

15. A pharmaceutical composition comprising the compound according to claim 13 or a pharmaceutically acceptable salt thereof and a pharmaceutically acceptable carrier.

16. A method of treatment to suppress one or more hormones selected from the group consisting of luteinizing hormone, follicle-stimulating hormone, estradiol and testosterone in a subject in need thereof, the method comprising administering to the subject an effective amount of a glycine transporter-1 inhibitor of formula I 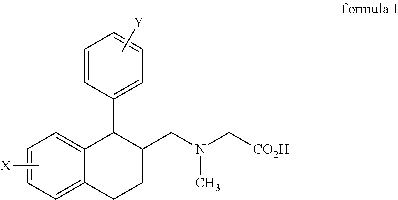
wherein
X is 1-3 substituents selected from H, halogen, methyl, methoxy, trifluoromethyl and trifluoromethoxy and
Y is 1-3 substituents selected from H, methyl and halogen
or a pharmaceutically acceptable salt thereof.

17. The method according to claim 16, wherein the hormone is luteinizing hormone.

18. The method according to claim 16, wherein the hormone is follicle-stimulating hormone.

19. The method according to claim 16, wherein the hormone is estradiol.

20. The method according to claim 16, wherein the hormone is testosterone.

21. The method according to claim 16, wherein the subject is a female human.

22. The method according to claim 16, wherein the treatment is part of a contraceptive regimen.

23. The method according to claim 16, wherein the treatment is for hypersexuality.

24. The method according to claim 16, wherein the compound of formula I is N-methyl-N-[[(1R,2S)-1,2,3,4-tetrahydro-6-methoxy-1-phenyl-2-naphthalenyl]methyl glycine or a pharmaceutically acceptable salt thereof.
